(19) United States
(12) Patent Application Publication
(10) Pub. No.: US 2022/0220043 A1
(43) Pub. Date: Jul. 14, 2022

(54) METHOD FOR RADIOIODINATION OR RADIOASTATINATION OF A BIOMOLECULE

(71) Applicants: INSTITUT NATIONAL DE LA SANTE ET DE LA RECHERCHE MEDICALE (INSERM), PARIS (FR); Université de Nantes, Nantes (FR); CHU Nantes, Nantes (FR); Centre National de la Recherche Scientifique, Paris (FR)

(72) Inventors: Marion BERDAL, Nantes (FR); François GUERARD, Nantes (FR); Jean-François GESTIN, Nantes (FR); Alain FAIVRE-CHAUVET, Nantes (FR)

(21) Appl. No.: 17/608,318

(22) PCT Filed: Apr. 28, 2020
(86) PCT No.: PCT/EP2020/061756

§ 371 Date: Nov. 2, 2021

(30) Foreign Application Priority Data
May 2, 2019 (EP) 19305563.9

(51) Int. Cl. C07B 59/00; C07K 16/28; C07F 5/02

(52) U.S. Cl. CPC C07B 59/008; C07B 2200/05; C07F 5/025; C07K 16/2803

(57) ABSTRACT

The present invention relates to a method for radioiodination or radioastatination of a biomolecule such as proteins and antibodies by reacting a biomolecule carrying a hetero(aryl) boronic acid group with a radioiodide or astatide salt, in the presence of a catalyst and a ligand, in a buffer solution, in order to obtain a radioiodo- or astatolabeled biomolecule. The method of the invention is thus a single step method easy to be implemented and efficient for both radioiodination and radioastatination of antibodies.

The present invention concerns a method for radioiodination or radioastatination of a biomolecule. It also concerns biomolecules carrying a (hetero)aryl boronic acid group, used as intermediate products.

Heavy radiohalogens astatine and iodine have been increasingly studied over io the past decades for therapeutic or diagnostic purpose in nuclear medicine. The most relevant iodine radioisotopes ¹²³|(γ⁺, t_{1/2}2=13.2 hours), 124|(β⁺, t_{1/2}=4.18 days), 125|(γ⁻, Auger e⁻, t_{1/2}=59.4 days) and ¹³¹ |(β⁻ and γ, t_{1/2}=8 days) can be used for imaging and/or therapy depending on the radiation they emit upon decay, whereas ²¹¹At (t_{1/2}7.2 h, α-emitter) is a most promising isotope for the treatment of small is cell clusters or isolated tumor cells, and ²⁰⁹At may be of use in imaging (t_{1/2}=5.4 h, γ emitter). The radioiodination strategy of relevant peptides and proteins has long been the direct electrophilic substitution on tyrosine. Despite the advantage of being a fast and simple procedure, this method exhibits limits for in vivo applications due to rapid deiodination that leads to radioiodine activity uptake in non-targeted organs (especially in thyroid and stomach). Consequently, more stable labeling strategies based on the use of a radioiodinated agent for acylation of lysine residues have been developed since then to overcome this issue (Garg, P. K., Alston, K. L. & Zalutsky, M. R. Catabolism of radioiodinated murine monoclonal antibody F(ab′)2 fragment labeled using N-succinimidyl 3-iodobenzoate and lodogen methods. Bioconjugate Chem. 6, 493-501 (1995); and Kim, E. J., Kim, B. S., Choi, D. B., Chi, S.-G. & Choi, T. H. Enhanced tumor retention of radioiodinated anti-epidermal growth factor receptor antibody using novel bifunctional iodination linker for radioimmunotherapy. Oncology Reports 35, 3159-3168 (2016)). The lack of sufficient stability of direct electrophilic labeling with astatine is even more marked (Visser, G. W. M., Diemer, E. L. & Kaspersen, F. M. The preparation and stability of astatotyrosine and astato-iodotyrosine. Int. J. Appl Radiat. Isot. 30, 749-752 (1979)), and in this case, the use of an astatinated prosthetic group is essential to carry out any in vitro or in vivo experimentation. Thus, several astatinated prosthetic groups have also been developed for conjugation to amino groups of lysine residues in order to obtain sufficient label stability for biological experimentations (Zalutsky, M. R., Garg, P. K., Friedman, H. S. & Bigner, D. D. Labeling monoclonal antibodies and F(ab′)2 fragments with the alpha-particle-emitting nuclide astatine-211: preservation of immunoreactivity and in vivo localizing capacity. Proc. Natl. Acad. U.S.A 86, 7149-7153 (1989); and Choi, J., Vaidyanathan, G., Koumarianou, E., Kang, C. M. & Zalutsky, M. R. Astatine-211 labeled anti-HER2 5F7 single domain antibody fragment conjugates: radiolabeling and preliminary evaluation. Nucl Med Biol 56, 10-20 (2018). The most used prosthetic groups to date are N-succinimidyl-3-[*I]iodobenzoate (NSIB) or N-succinimidyl-3-[²¹¹At]astatobenzoate ([²¹¹At]SAB) which are comprised of an activated ester for conjugation to the lysine residue of proteins. The conjugation step requires a mildly basic aqueous solution (pH≈8.5) to make the amino group sufficiently reactive with the activated ester. However, competitive hydrolysis of the ester also occurs at this pH, leading to the production of the inactive benzoate side product and to suboptimal conjugation yields. In the most favorable cases, relatively good conjugation yields can be obtained by this approach but a minimum protein concentration of 4-5 mg/mL is necessary to favor lysine conjugation over competitive hydrolysis, a concentration that is not always compatible with antibodies at this pH due to aggregation and precipitation issues and which also limits the achievable specific activity.

To avoid the radioactive loss observed during the bioconjugation step, strategies have been developed which consist in coupling a non-radioactive organotin precursor in the first step (either on lysine or on cysteine residues) and then perform the electrophilic radiolabeling directly on the obtained pre-modified biomolecule (Lindegren, S. et al. Direct procedure for the production of 211At-labeled antibodies with an epsilon-lysyl-3-(trimethylstannyl)benzamide immunoconjugate. J. Nucl. Med 49, 1537-1545 (2008); and Aneheim, E. et al. Synthesis and Evaluation of Astatinated N-[2-(Maleimido)ethyI]-3-(trimethylstannyl)benzamide Immunoconjugates. Bioconjugate Chem. 27, 688-697 (2016)). While this strategy is efficient for radiolabeling the reported antibodies with ²¹¹At, this approach exhibits several limits. First of all, it is not applicable for labeling with iodine radioisotopes. Indeed, the precursor used, an organotin compound, requires the halogen in the X+ form (I+, At+) to perform the electrophilic destannylation reaction that forms the halogen-protein bond. However, under these conditions, L also forms an unstable bond with tyrosines as described above. Second, the At+ species required for astatination by electrophilic approach is quite unstable: several other oxidized species of astatine can form in oxidizing media, and the At+ species tends to evolve over time into the reduced species At due to solvent radiolysis. Consequently, the use of electrophilic approaches to label molecules with ²¹¹At often leads to inconsistent results that may hamper industrial and clinical transfer.

The aim of the present invention is thus to provide a late stage radiolabeling approach of biomolecule that could be used for both iodine and astatine radioisotopes.

Another aim of the present invention is to provide a method for both the radioiodination and the radioastatination of a biomolecule, such as an antibody, being easy to be implemented, and carried out in mild conditions, in particular in an aqueous medium.

Therefore, the present invention relates to a method for radioiodination or radioastatination of a biomolecule comprising a step of reacting a biomolecule carrying a hetero(aryl) boronic acid group with a radioiodide or astatide salt, in the presence of a catalyst and a ligand, in a buffer solution, in order to obtain a radioiodo- or astatolabeled biomolecule.

The method of the invention thus involves a single step that may be carried out in aqueous medium, such as water.

Within the present invention, the term “(hetero)aryl” includes both terms “aryl” and “heteroaryl”.

Within the present invention, the term “aryl” refers to an aromatic monocyclic, bicyclic, or tricyclic hydrocarbon ring system, wherein any ring atom capable of substitution may be substituted by a substituent. Examples of aryl moieties include, but are not limited to, phenyl, naphthyl, and anthracenyl. The preferred substituents on aryl groups are amino, amine, alkoxy, halo, perfluoroalkyl such as CF₃, heterocyclyl, amide, and ester.

Within the present invention, the term “heteroaryl” refers to a 5- to 10-membered aromatic monocyclic or bicyclic group containing from 1 to 4 heteroatoms selected from O, S or N. By way of examples, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl and isoquinolinyl groups.

By way of a heteroaryl comprising 5 to 6 atoms, including 1 to 4 nitrogen atoms, mention may in particular be made of the following representative groups: pyrrolyl, pyrazolyl, 1, 2, 3-triazolyl, 1, 2, 4-triazolyl, tetrazolyl and 1, 2, 3-triazinyl.

Mention may also be made, by way of heteroaryl, of thiophenyl, oxazolyl, furazanyl, 1, 2, 4-thiadiazolyl, naphthyridinyl, quinoxalinyl, phthalazinyl, imidazo[1, 2-a]pyridine, imidazo[2, 1-b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1, 2, 4-triazinyl, indolizinyl, isoxazolyl, isoquinolinyl, isothiazolyl, purinyl, quinazolinyl, quinolinyl, isoquinolyl, 1,3, 4-thiadiazolyl, thiazolyl, isothiazolyl, carbazolyl, and also the corresponding groups resulting from their fusion or from fusion with the phenyl nucleus.

Examples of heteroaryl moieties include, but are not limited to, pyridinyl io moieties.

According to an embodiment, the iodide or astatide salt has the formula A+X⁻, A⁺ being a monovalent cation selected among sodium, potassium, cesium, tetraalkylammonium, and tetraalkylphosphonium, and X⁻being iodide or astatide. Preferably, X⁻is ¹²³|⁻, ¹²⁴|⁻, ¹²⁵|⁻, ¹³¹|⁻ or ²¹¹At⁻. More preferably, X⁻is ¹²⁵|⁻or ²¹¹At⁻.

According to an embodiment, the catalyst is chosen from the group consisting of: Cu₂O, Cu(CO₂CH₃)₂, Cu(OCOCF₃)₂H₂O, Cu(CH₃CN)₄OTf, and Cu(OTf)₂pyr₄.

Preferably, the catalyst is Cu(OTO₂pyr_{4.}

According to an embodiment, the ligand is chosen from the group consisting of: 1,10-phenanthroline, 4, 7-dihydroxyphenanthroline, bathophenanthorlinedisulfonic acid disodium salt hydrate, dichloro (1,10-phenanthroline) copper II, and 3, 5, 7, 8-tetramethyl-1,10-phenanthroline.

Preferably, the ligand is 1,10-phenanthroline.

According to an embodiment, the buffer solution is chosen from the group consisting of: carbonate buffer, borate buffer, 4-(2-hydroxyethyl)-1-piperazineethanesulfonic acid (HEPES) buffer, tris(hydroxymethyl)aminomethane (TRIS) buffer, acetate buffer, 2-(N-morpholino)ethanesulfonic acid (MES) buffer, and 3-(N-morpholino)propanesulfonic acid (MOPS) buffer.

Preferably, the buffer solution is TRIS buffer.

According to an embodiment, the pH of the buffer solution is comprised between 3 and 8.5, and is preferably equal to 6.

According to an advantageous embodiment, the method of the invention comprises a step of reacting a biomolecule carrying a hetero(aryl) boronic acid group with a radioiodide or astatide salt, in the presence of Cu(OTO₂pyr₄ as catalyst and 1,10-phenanthroline as ligand, in a TRIS buffer solution, in order to obtain a radioiodo- or astatolabeled biomolecule.

According to an embodiment, the biomolecule is chosen from the group consisting of: proteins, antibodies, fragments of antibodies, antibody constructs like minibodies, diabodies etc. . . . resulting from antibody engineering, as recombinant proteins, and synthetic peptides selected to bind target cells, e.g., but not limited to, affibodies or affitins.

Preferably, the biomolecule is an antibody.

According to an embodiment, the biomolecule carrying a hetero(aryl) boronic is acid group is a biomolecule comprising a group having the following formula (I):

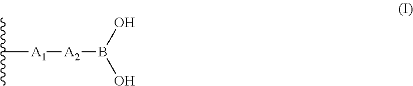

wherein:

A₁ is a linker, and

A₂ is a (hetero)aryl group, optionally substituted with at least one substituent.

According to an embodiment, one of the terminal atoms of the linker A₁ is linked to A₂ and the other one is linked to an atom of the biomolecule.

As linker A₁, the followings may be mentioned: (C₁-C₆)alkylene groups, —O—, —C(═O)—, —O—C(═O)—, —C(═O)—O—, —NRₐ—, —C(═O)—NRₐ—, —NR—, —C(═O)—, Rₐ being a hydrogen atom or a (C₁-C₆)alkyl group.

According to an embodiment, A₁ is chosen from or may contain one of the following radicals:

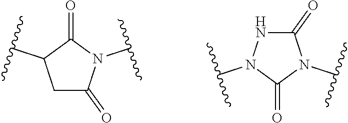 [SMILES: *C1CC(=O)N(*)C1=O ; *n1[nH]c(=O)n(*)c1=O]

According to an embodiment, A₁ may be represented by the formula -L₁-L₂-, wherein:

L₁ has one of the following formulae:

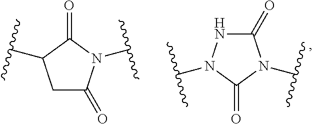 [SMILES: *C1CC(=O)N(*)C1=O ; *n1[nH]c(=O)n(*)c1=O]

and

L₂ is chosen from the group consisting of: —(C₁-C₆)alkylene, —NRₐ—C(═O)—, in particular —NHC(═O)—, —C(═O)—, —C(═O)—(C₁-C₆)alkylene-C(═O)—, —(C₁-C₆)alkylene-NRₐ-C(═O)—, Rₐ being as defined above.

Preferably, L₂ is a —(CH₂)₂—NH—C(═O)— group, the —C(═O) group being linked to A₂ a defined above.

According to an embodiment, the linker A₁ may be a trivalent radical such as a CH group able to bind to two atoms of the biomolecule.

According to an embodiment, A₁ contains a group L₃ obtainable by click chemistry. These click chemistry reactions include in particular the cycloadditions of unsaturated compounds, among which one may cite the Diels-Alder reactions between a dienophile and a diene, and especially also the azide-alkyne 1,3-dipolar cycloadditions, and preferably the copper-catalyzed azide-alkyne cycloaddition (CuAAC).

Preferably, L₃ is obtained by the reaction between two reactive functions, said reaction being selected from the group consisting of:

the reaction between an azide and an alkyne,

the reaction between an aldehyde or ketone and an hydrazide,

the reaction between an aldehyde or ketone and an oxyamine,

the reaction between an azide and a phosphine,

the reaction between an alkene and a tetrazine,

the reaction between an isonitrile and a tetrazine, and

the reaction between a thiol and an alkene (thiol-ene reaction).

the reaction between a tetrazine and a trans-cyclooctene

More preferably, L₃ is selected from the group consisting of the following radicals:

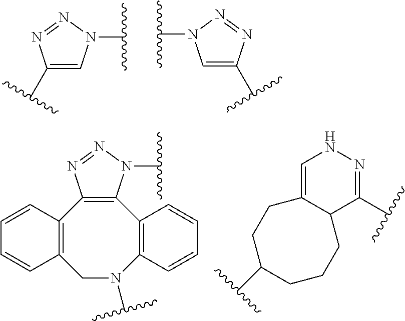

According to an embodiment, A₁ is represented by the formula -L₄-L₃-(L₅)ᵢ, wherein:

i is 0 or 1;

L₃ is a defined above; and

L₄ is chosen from the group consisting of: —O—, —C(═O)—O—, —O—C(═O), —C(═O)—NRₐ—, —NRₐ—C(═O)—, —C(═O)—(C₁-C₆)alkylene, —C(═O)—(C₁-C₆)alkylene-C(═O)—, and —C(═O)—NRₐ—(C₁-C₃₅)alkylene-O—(C₁-C₆)alkylene-NRₐ—C(═O)—O—,

L₅ is chosen from the (C₁-C₆)alkylene radicals and is preferably CH₂.

Preferably, A₁ is a —C(═O)— group.

As mentioned above, A₂ is a (hetero)aryl group, preferably a phenyl or pyridinyl group, optionally substituted with at least one substituent. The following substituents may be mentioned for example: amino, hydroxyl, thiol, oxo, halogen, (C₁-C₆)alkyl, (C₁-C₆)alkoxy, (C₁-C₆)alkylthio, (C₁-C₆)alkylamino, aryloxy, aryl(C₁-C₆)alkoxy, cyano, halo(C₁-C₆)alkyl, carboxyl and carboxy(C₁-C₆)alkyl.

Within the present application, the term “a halogen atom” means: a fluorine, a chlorine, a bromine or an iodine.

Within the present application, the term “a haloalkyl group” means: an alkyl group as defined above, in which one or more of the hydrogen atoms is (are) replaced with a halogen atom. By way of example, mention may be made of fluoroalkyls, in particular CF₃ or CHF2.

Within the present application, the term “an alkoxy group” means: an —O-alkyl radical where the alkyl group is as previously defined. By way of examples, mention may be made of —O—(C₁-C₄)alkyl groups, and in particular the —O-methyl group, the —O-ethyl group as —O—C₃alkyl group, the —O-propyl group, the —O-isopropyl group, and as —O—C₄alkyl group, the —O-butyl, —O-isobutyl or —O-tert-butyl group.

Within the present application, the term “an alkylthio” means: an —S-alkyl group, the alkyl group being as defined above.

Within the present application, the term “an alkylamino” means: an —NH-alkyl group, the alkyl group being as defined above. is Within the present application, the term “an aryloxy” means: an —O—aryl group, the aryl group being as defined above.

Within the present application, the term “an arylalkoxy” means: an aryl-alkoxy-group, the aryl and alkoxy groups being as defined above.

Within the present application, the term “a carboxyalkyl” means: an HOOC-alkyl-group, the alkyl group being as defined above. As examples of carboxyalkyl groups, mention may in particular be made of carboxymethyl or carboxyethyl.

Within the present application, the term “a carboxyl” means: a COOH group. Within the present application, the term “an oxo” means: “═O”. When an alkyl radical is substituted with an aryl group, the term “arylalkyl” or “aralkyl” radical is used. The “arylalkyl” or “aralkyl” radicals are aryl-alkyl-radicals, the aryl and alkyl groups being as defined above. Among the arylalkyl radicals, mention may in particular be made of the benzyl or phenethyl radicals.

The abovementioned “alkyl” substituent may also be substituted with one or more substituents. Among these substituents, mention may be made of the following groups: amino, hydroxyl, thiol, oxo, halogen, alkyl, alkoxy, alkylthio, alkylamino, aryloxy, arylalkoxy, cyano, trifluoromethyl, carboxy or carboxyalkyl.

Preferred substituents of the (hetero)aryl group, in particular phenyl or pyridinyl group, are halogen atoms.

Other substituents of said (hetero)aryl group, in particular phenyl or pyridinyl group, are the followings:

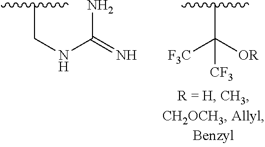

According to a preferred embodiment, the hetero(aryl) boronic acid group is a biomolecule comprising a group having the following formula (1-1):

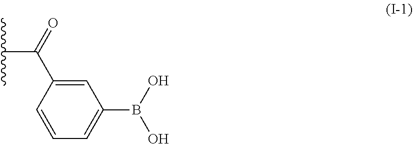 [SMILES: *C(=O)c1cccc(B(O)O)c1]

According to an embodiment, the radioiodo- or astatolabeled biomolecule as obtained comprises a group having the following formula (II):

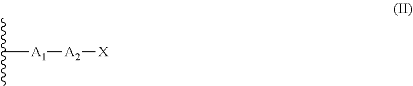

wherein: A₁ and A₂ are as defined above in formula (I), and X is ¹²³|, ¹²⁴|, ¹²⁵|, ¹³¹| or ²¹¹At, preferably ¹²⁵| or ²¹¹At.

According to a preferred embodiment, the radioiodo- or astatolabeled biomolecule as obtained comprises preferably a group having the following formula (II-1):

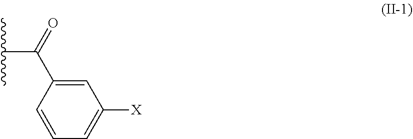

The present invention also relates to a method as defined above, for the preparation of a radioiodo- or astatolabeled biomolecule having the following formula (III):

A-A₁-A₂—X (III)

A being a biomolecule as defined above,

A₁ and A₂ being as defined above in formula (I), and X being ¹²³|, ¹²⁴|, ¹²⁵|, ¹³¹| or ²¹¹ At, preferably ¹²⁵| or ²¹¹At, said radioiodo- or astatolabeled biomolecule having preferably the following formula (III-1):

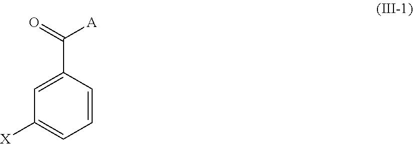

The present invention relates to a biomolecule carrying a (hetero)aryl boronic acid group, wherein the (hetero)aryl boronic acid group is linked to said biomolecule through an (hetero)aromatic group, in particular an arylene group, more preferably a phenylene group.

According to an embodiment, the biomolecule carrying a (hetero)aryl boronic acid group as defined above comprises a group having the following formula (I):

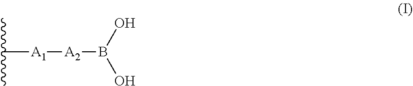

A₁ and A₂ being as defined above in formula (I), said hetero(aryl) boronic acid group being preferably a biomolecule comprising a group having the following formula (I-1):

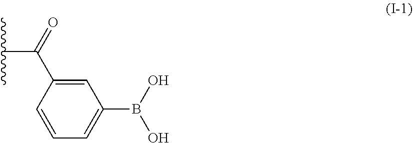 [SMILES: *C(=O)c1cccc(B(O)O)c1]

According to an embodiment, the biomolecule carrying a (hetero)aryl boronic acid group as defined above comprises a group having the following formula (IV):

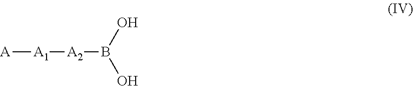

A being a biomolecule, and

A₁ and A₂ being as defined in above in formula (I).

According to an embodiment, the biomolecule carrying a (hetero)aryl boronic acid group as defined above is an antibody.

### EXAMPLES

### Example 1

### Preparation of (3-(N-hydroxysuccinimidyl)carbonyl) phenyl)boronic acid 2.

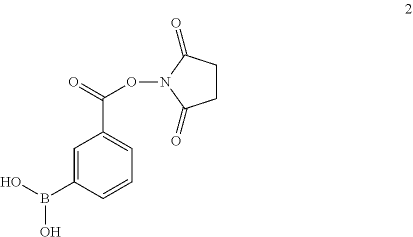 [SMILES: O=C(ON1C(=O)CCC1=O)c1cccc(B(O)O)c1]

To a solution of 3-boronobenzoic acid (3 mmol, 1 eq) in DMF (25 mL) was added EDCI (4.5 mmol, 1.5 eq), N-hydroxysuccinimide (4.5 mmol, 1.5 eq) and triethylamine (27 mmol, 9 eq). The solution was stirred for 28 h. The solvent was evaporated in vacuo and the obtained residue was dissolved in CH₂Cl₂. 1N HCl was then added to the mixture and the aqueous layer was extracted three times with CH₂Cl₂. The organics layers were dried over MgSO₄, filtered and the solvent removed under reduced pressure. The crude product was then purified on a silica gel column using MeOH (0% to 1.5%) in CH₂Cl₂ to provide 2 as a white solid (123 mg, 45% yield). ¹H NMR (400 MHz, DMSO) δ 8.53 (s, 1H), 8.42 (s, 2H), 8.20 (d, J=7.4 Hz, 1H), 8.12 (d, J=8.0 Hz, 1H), 7.63 (t, J=7.7 Hz, 1H), 2.90 (s, 4H); ¹³C NMR (100 MHz, DMSO) 6 170.33, 162.07, 140.92, 135.55, 131.41, 128.60, 123.79, 39.51, 25.53.

### Example 2

### Bioconjugation of Arylboronic Acid 2—Preparation of a Biomolecule carrying an Arylboronic Acid Group

The late stage radiolabeling of antibodies was assessed on anti-CD22 monoclonal antibody (mAb) and on the 9E7.4 IgG, an mAb directed against murine CD138 for targeting multiple myeloma cells (Fichou, N. et al. Single-dose anti-CD138 radioimmunotherapy: bismuth-213 is more efficient than lutetium-177 for treatment of multiple myeloma in a preclinical model. Front. Med. 76 (2015). doi:10.3389/fmed.2015.00076).

For this, a bifunctional aBA (arylboronic acid), (3-(N-hydroxy-succinimidyl)carbonyl)phenyl)boronic acid 2 as mentioned above was conjugated to these mAbs via their lysine side chains:

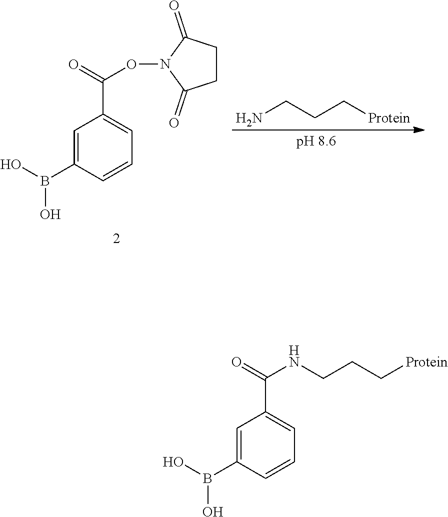

To a 5 mg/mL anti-CD22 or 9E7.4 0.3 M Borate buffer solution (pH 8.6) was added 5 to 50 equivalents of 2 in DMF. The solution was stirred at room temperature for 75 min upon which the unconjugated acid was removed by ultracentrifugation with 30K centrifugal filters (Merck) using the buffer needed for radiolabeling. Concentration was set at about 6 mg/mL.

The corresponding compounds are named aBA-anti-CD22 and aBA-9E7.4 (biomolecule carrying a arylboronic group).

### Example 3

### Radioiodination and Astatination of aBA-anti-CD22 and aBA-9E7.4

Na[¹²⁵|]| was obtained commercially from Perkin Elmer in 10⁻⁵ M NaOH solution with a volumic acitivity of 50 μCi/pL (1.85 MBq/pL). ²¹¹At was produced at the Arronax cyclotron facility using the ²⁰⁹Bi(α, 2n)²¹¹At reaction and recovered from the irradiated target in chloroform using a dry-distillation protocol adapted from the procedure previously reported by Lindegren et al. (Lindegren, S., Back, T. & Jensen, H. J. Dry-distillation of astatine-211 from irradiated bismuth targets: a time-saving procedure with high recovery yields. Appl. Radiat. Isot. 55, 157-160 (2001)). Na[²¹¹At]At was then obtained by reducing to dryness the chloroformic astatine solution under a gentle stream of nitrogen and dissolving the dry residue in an appropriate volume of CH₃CN followed by the same volume of a 0.125 to 10 mg/mL sodium sulfite solution.

To aBA-anti-CD22 or aBA-9E7.4 in 0.5 M TRIS buffer at pH=6.0 (40 μL) at a concentration between 6 mg/mL and 0.75 mg/mL were added Cu(OTO₂pyra in 0.5 M TRIS buffer pH 6/DMF (1:1) (5 μL), 1.10-phenanthroline in 0.5 M TRIS buffer pH 6/DMF (1:1) (2.5 μL), and Na[¹²⁵|]| or Na[²¹¹At]At (5 μL). After 30 min of incubation at room temperature, the radiolabeling yield was assessed by elution of an aliquot deposited on an ITLC-SG strip (MeOH as eluent), and integration of the strip using a Cyclone phosphorimager scanner (Perkin Elmer). Purification was performed by gel filtration on a Sephadex G-25 resin loaded column (PD-10, GE healthcare) using PBS as eluent, affording the purified radiolabeled antibody with a >99% radiochemical purity as assessed by ITLC-SG.

Immunoreactivity Assay of 9E7.4 Antibody

The immunoreactive fraction of [¹²⁵|]aBA-9E7.4 and [²¹¹At]aBA-9E7.4 was determined using magnetic beads (Pierce, Thermo Scientific) labeled with a 40 amino acids peptide recognized by the 9E7.4 antibody according to the supplier's protocol. 0.1 picomole of radiolabeled aBA-9E7.4 was incubated for 15 min at room temperature with 20 μL of coated magnetic beads (10 mg/mL). Using a magnetic rack, supernatants containing non-reactive antibodies and magnetic beads were is collected separately and the radioactivity in each fraction was measured in a gamma counter.

### Example 4

### Optimization on anti-CD22.

5 to 50 equivalents of 2 were incubated with anti-CD22. The number of aBAs conjugated per anti-CD22 in the resulting aBA-anti-CD22 was assessed by mass spectrometry. Radioiodination and astatination efficiency was evaluated using the optimal conditions determined with model compound 1 (4-chlorobenzeneboronic acid)(pH=6, 10% DMF, catalyst and ligand concentration=10 mM). Under these conditions high RCYs were obtained at 10 eq of 2 or more, however partial precipitation of aBA-anti-CD22 was observed during radiolabeling when 25 or more eq of 2 where conjugated to anti-CD22 (table 1). Consequently, it was determined that conjugating 10 equivalents aBA to anti-CD22 resulted in the optimal result with 93% radioiodination and 94% astatination and no precipitation observed.

TABLE 1
Influence of equivalents of 2 conjugated to anti-CD22 on radioiodination
and astatination RCY and on the precipitation of the resulting
aBA-anti-CD22 in the radiolabeling step.^{a}

| Equivalents of 2 in | | RCY (%) | | IgG |
|---|---|---|---|---|
| bioconjugation step | aBAs/antibody^{b} | ¹²⁵I | ²¹¹At | precipitation |
| 5 | — | 58 | 49 | No |
| 10 | 4.6 ± 1.1 | 93 | 94 | No |
| 25 | — | 94 | 95 | Yes |
| 50 | — | 98 | 98 | Yes |

^{a}Standard conditions: aBA-anti-CD22 (32 μM), Cu(OTf)₂Pyr₄ (10 mM), 1,10-phenanthroline (10 mM), Na[¹²⁵I]I or Na[²¹¹At]At (1-5 MBq), 30 min, 23° C. in 0.5M TRIS buffer/DMF (92.5:7.5).
^{b}Determined by mass spectrometry (n = 2)

### Example 5

### Radiolabeling and in vitro Evaluation of 9E7.4.

Optimal conditions for bioconjugation and radiolabeling determined with anti-CD22 were used with 9E7.4. Namely, 10 equivalents of compound 2 were used in the bioconjugation step in order to generate aBA-9E7.4. Mass spectrometry analyses indicated a ratio of 4.09 ±0.05 aBA/antibody (n=2). 5 mM catalyst and ligand concentrations were used for radioiodination whereas 2.5 mM were used for astatination. RCYs remained high (Table 2). After purification, the immunoreactive fraction was 94% after radioiodination and 86% after astatination. These results are similar if not better to the conventional two step radiolabeling procedure on the same mAb (86% for radioiodination and astatination as reported previously (Guerard, F. et al. Bifunctional aryliodonium salts for highly efficient radioiodination and astatination of antibodies. Bioorg. Med. Chem. 25, 5975-5980 (2017))), showing that the protein retained its activity against CD138.

TABLE 2
Radioiodination and astatination of aBA-9E7.4 IgG

| | | Immunoreactive |
|---|---|---|
| Radionuclide | RCY (%) | fraction (%) |
| ¹²⁵I^{a} | 78 | 94 |
| ²¹¹At^{b} | 84 | 86 |

^{a}aBA-9E7.4 (32 μM), Cu(OTf)₂Pyr₄ (5 mM), 1,10-phenanthroline (5 mM), Na[¹²⁵I]I (1-5 MBq), 30 min, 23° C. in 100 μL 0.5M TRIS buffer/DMF (9:1).
^{b}modified 9E7.4 (32 μM), Cu(OTf)₂Pyr₄ (2.5 mM), 1,10-phenanthroline (2.5 mM), Na[²¹¹At]At (1-5 MBq), 30 min, 23° C. in 0.5M TRIS buffer/DMF (92.5:7.5).

### Example 6

### Optimization of the Antibody Concentration

The last step was to reduce the concentration of antibody in the radiolabeling to improve the specific activity (Tables 3 and 4).

TABLE 3
Influence of the concentration of antibody on radioastatination
RCY of aBA-anti-CD22, anti-CD22^{a}

| | |
|---|---|
| mAb | RCY (%) |

| concentration | aBA-anti- | Anti- |
|---|---|---|
| (mg/mL) | CD22 | CD22 |
| 4.8 | 93 ± 2.5^{b} | 6.7 ± 2.3^{b} |
| 3.6 | 92 ± 2.1^{c} | 5.8 ± 1.0^{c} |
| 3.0 | 87 ± 5.9^{b} | 5.7 ± 0.5^{b} |
| 2.4 | 74 ± 9.2^{c} | 4.1 ± 0.2^{c} |
| 1.2 | 65^{d} | 3.1^{d} |

^{a}Standards conditions: Cu(OTf)₂Pyr₄ (2.5 mM), 1,10-phenanthroline (2.5 mM), Na[²¹¹At]At (1-5 MBq), 30 min, 50 mL, 23° C. in TRIS buffer/DMF 92.5:7.5).
^{b}n = 3.
^{c}n = 2.
^{d}n = 1.

TABLE 4
Influence of the concentration of antibody on radioiodination
and radioastatination RCY of aBA-9E7.4 and 9E7.4^{a}

| | RCY (%) | | | |
|---|---|---|---|---|
| mAb | ¹²⁵I^{a} | | ²¹¹At^{b} | |
| concentration | aBA-anti- | Anti- | aBA-anti- | Anti- |
| (mg/mL) | CD138 | CD138 | CD138 | CD138 |
| 4.8 | 93 | 1.2 | 95 | 38 |
| 3.6 | 95* | 1.5 | 94 | 26 |
| 3.0 | 87 | — | 92 | 30 |
| 2.4 | 81 ± 2^{e} | — | 94 | 26 |
| 1.8 | 75 | — | 91.5 ± 1.5^{e} | 14.5 ± 2.5^{e} |
| 1.8 | 89^{d} | — | — | — |
| 1.2 | 2.1 | — | 86 | 13 |
| 0.6 | — | — | 62.5 ± 9.5^{e} | 6.55 ± 0.65^{e} |
| 0.6 | — | — | 89^{c} | 9^{c} |

^{a}Cu(OTf)₂Pyr₄ (5 mM), 1,10-phenanthroline (5 mM), Na[¹²⁵I]I (0.5-2 MBq), 30 min, 50 mL, 23° C. in TRIS buffer/DMF 92.5:7.5).
^{b}Cu(OTf)₂Pyr₄ (2.5 mM), 1,10-phenanthroline (2.5 mM), Na[²¹¹At]At (1-5 MBq).
^{c}reaction lasted 60 min instead of 30 min.
^{d}catalyst (2.5 mM, ligand (2.5 mM), reaction lasted 90 min instead of 30 min.
^{e}n = 2

Results indicate that antibody concentration can be decreased significantly, especially in the case of 9E7.4 IgG, without decreasing RCY.

Interestingly, high RCYs were also obtained for lower concentrations (0.6 mg/mL) when reaction time was extended to one hour. Thus this labeling strategy appears interesting to increase specific activity in comparison with conventional approaches.

### Example 7

### Biodistribution study of [¹²⁵1]9E7.4 and [²¹¹At]9E7.4 produced in one step from aBA-9E7.4 and comparison with conventional two-step approach from 9E7.4.

To Balb/c mice were injected in the flank 200, 000 MOPC 315 cells (CD138+) and biodistribution studies were performed 17 days after cells injection.

[¹²⁵1]9E7.4 and [²¹¹At]9E7.4 were obtained in one step as described in example 5 or in two steps via the preparation of N-succinimidyl-3-[²¹¹At]astatobenzoate from an iodonium salt precursor as described previously (Guerard, F. et al. Bifunctional aryliodonium salts for highly efficient radioiodination and astatination of antibodies. Bioorg. Med. Chem. 25, 5975-5980 (2017)).

Mice received 20 pg of [¹²⁵1]9E7.4 or [²¹¹At]9E7.4 obtained by each method and at least 3 animals were sacrificed 0.5 h, 1.5 h, 7 h, 14 h and 21 h after injection. Their tumors and organs were removed and weighed, and the radioactivity was counted using a y-counter. The results were expressed as percentage of injected dose per gram (% ID/g) except for the thyroid that was expressed as percentage of injected dose (% ID).

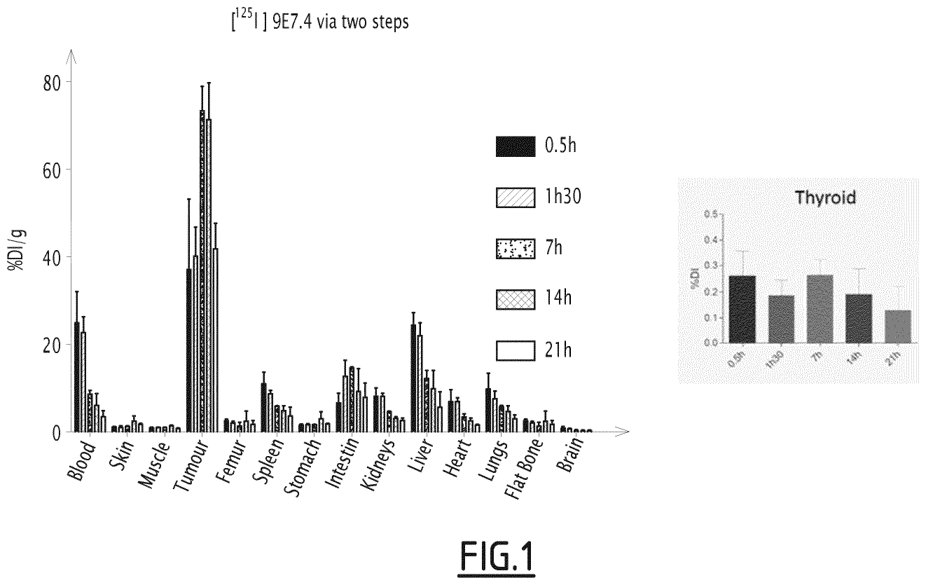

Results are shown in FIGS. 1-4:

FIG. 1. Biodistribution of [¹²⁵|]9E7.4 produced by the two-step method in mice grafted with MOPC 315 cells (n 3).

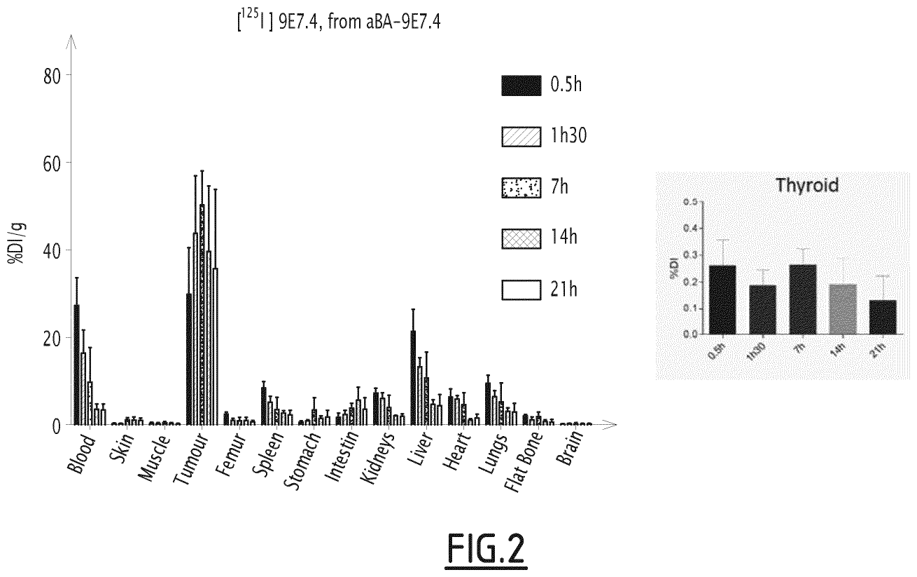

FIG. 2. Biodistribution of [¹²⁵|]9E7.4 produced in one step from aBA-9E7.4 in mice grafted with MOPC 315 cells (n≥3).

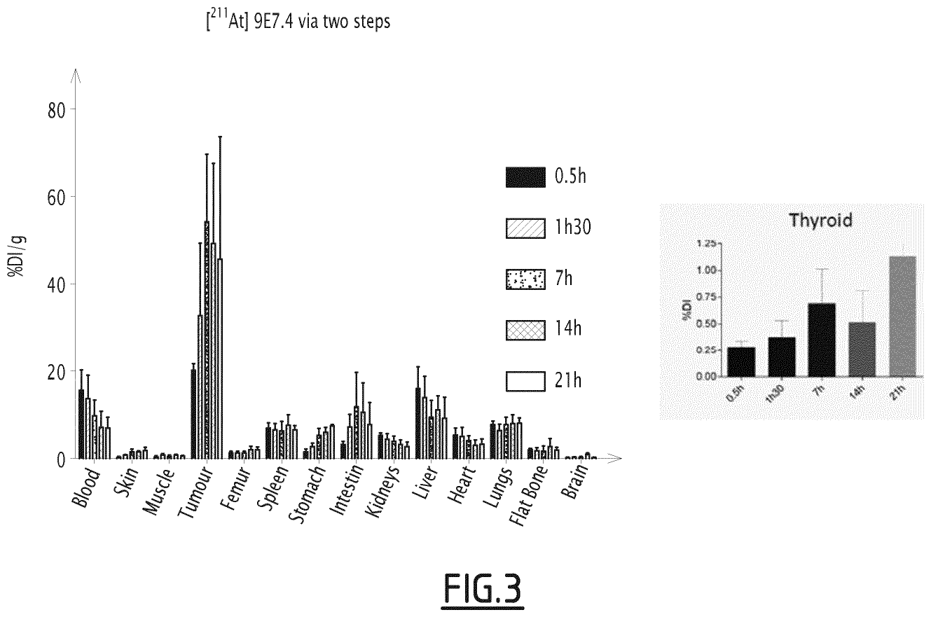

FIG. 3. Biodistribution of [²¹¹At]9E7.4 produced by the two-step method in mice grafted with MOPC 315 cells (n≥3).

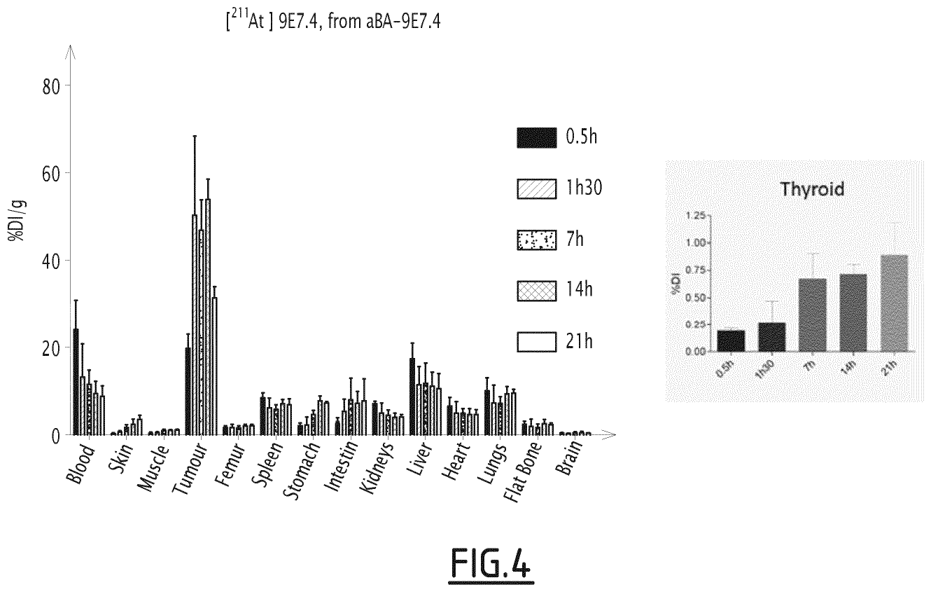

FIG. 4. Biodistribution of [²¹¹At]9E7.4 produced in one step from aBA-9E7.4 in mice grafted with MOPC 315 cells (n≥3).

Comparison of results obtained by both radiolabelling methods indicate that there is no significant difference in the pharmacokinetic behavior of the antibody.

The only major difference is observed for the tumor uptake of [¹²⁵|]9E7.4 obtained by the two-step approach (FIG. 1) that is higher than with the one-step approach (FIG. 2) but that is due to heterogeneity of tumor weigh in both groups, tumors exhibiting lower weights in the first case, resulting in higher %ID/mass ratio.

Another difference that may be noticed is the lower uptake in intestine with the one-step approach (FIGS. 2 and 4) compared to the two-step approach (FIGS. 1 and 3) indicating a better metabolic elimination resulting in more favorable dosimetry to the intestine of the radiolabelled antibody when labelled by the approach detailed in the present application in comparison with the two-step approach.

1. A method for radioiodination or radioastatination of a biomolecule comprising a step of reacting a biomolecule carrying a hetero(aryl) boronic acid group with a radioiodide or astatide salt, in the presence of a catalyst and a ligand, in a buffer solution, in order to obtain a radioiodo- or astatolabeled biomolecule.

2. The method of claim 1, wherein the iodide or astatide salt has the formula A⁺X⁻, wherein A⁺ is a monovalent cation selected among sodium, potassium, cesium, tetraalkylammonium, and tetraalkylphosphonium, and X⁻is iodide or astatide.

3. The method of claim 2, wherein X⁻ is 123|, ¹²⁴|, ¹²⁵|, ¹³¹|, or ²¹¹At⁻.

4. The method of claim 1, wherein the catalyst is selected from the group consisting of: Cu₂O, Cu(CO₂CH₃)₂, Cu(OCOCF₃)₂. H₂O, Cu(CH₃CN)₄OTf, and Cu(OTf)₂pyr₄.

5. The method of claim 1, wherein the ligand is selected from the group consisting of: 1,10-phenanthroline, 4,7-dihydroxyphenanthroline, bathophenanthorlinedisulfonic acid disodium salt hydrate, dichloro (1,10-phenanthroline) copper II, and 3,5,7,8-tetramethyl-1,10-phenanthroline.

6. The method of claim 1, wherein the buffer solution is selected from the group consisting of: carbonate buffer, borate buffer, HEPES buffer, TRIS buffer, acetate buffer, MES buffer, and MOPS buffer.

7. The method of claim 1, wherein the pH of the buffer solution is comprised between 3 and 8.5.

8. The method of claim 1, wherein the biomolecule is selected from the group consisting of: proteins, antibodies, fragments of antibodies, antibody constructs, as recombinant proteins, and synthetic peptides selected to bind target cells.

9. The method of claim 1, wherein the biomolecule carrying a hetero(aryl) boronic acid group is a biomolecule comprising a group having the following formula (I): 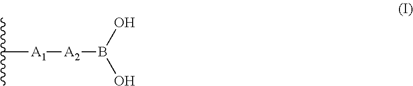
wherein:
A₁ is a linker, and
A₂ is a (hetero)aryl group, optionally substituted with at least one substituent, said hetero(aryl) boronic acid group being a biomolecule comprising a group having the following formula (I-1):

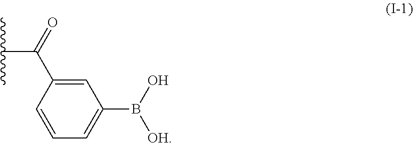 [SMILES: *C(=O)c1cccc(B(O)O)c1]

10. The method of claim 9, wherein the radioiodo- or astatolabeled biomolecule comprises a group having the following formula (II): 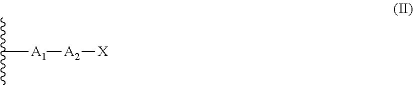
wherein X is ¹²³|, ¹²⁴‥, ¹²⁵|, ¹³¹| or ²¹¹At, said radioiodo- or astatolabeled biomolecule comprising a group having the following formula (II-1):

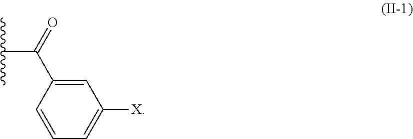

11. The method of claim 1, for the preparation of a radioiodo- or astatolabeled biomolecule having the following formula (III):
A-A₁-A₂-X (III)
wherein
A is a biomolecule,
A₁ is a linker,
A₂ is a (hetero)aryl group, optionally substituted with at least one substituent, said hetero(aryl) boronic acid group being a biomolecule comprising a group having the following formula (I-1): 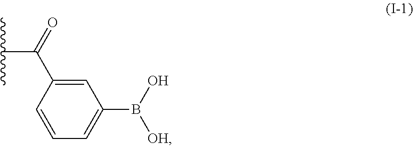 [SMILES: *C(=O)c1cccc(B(O)O)c1]
and X is ¹²³|, ¹²⁴|, ¹²⁵|, ¹³¹| or ²¹¹At,
said radioiodo- or astatolabeled biomolecule having the following formula (III-1):

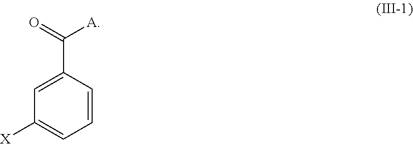

12. A biomolecule carrying a (hetero)aryl boronic acid group, wherein the (hetero)aryl boronic acid group is linked to said biomolecule through an (hetero)aromatic group.

13. The biomolecule carrying a (hetero)aryl boronic acid group of claim 12, which comprises a group having the following formula (I): 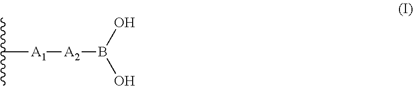
A₁ is a linker,
A₂ is a (hetero)aryl group, optionally substituted with at least one substituent, said hetero(aryl) boronic acid group being a biomolecule comprising a group having the following formula (I-1): 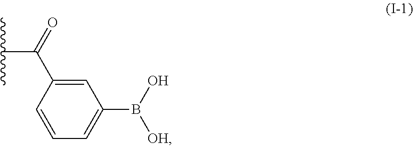 [SMILES: *C(=O)c1cccc(B(O)O)c1]
said hetero(aryl) boronic acid group being a biomolecule comprising a group having the following formula (I-1):

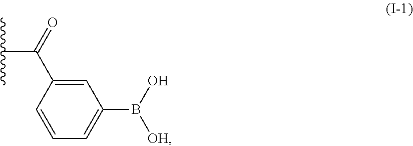 [SMILES: *C(=O)c1cccc(B(O)O)c1]

14. The biomolecule carrying a (hetero)aryl boronic acid group of claim 12, which comprises a group having the following formula (IV): 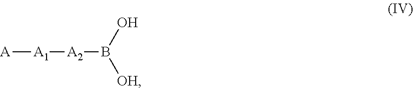
wherein
A is a biomolecule,
A₁ is a linker and
A₂ is a (hetero)aryl group, optionally substituted with at least one substituent, said hetero(aryl) boronic acid group being a biomolecule comprising a group having the following formula (I-1):

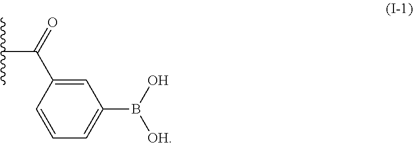 [SMILES: *C(=O)c1cccc(B(O)O)c1]

15. The biomolecule carrying a (hetero)aryl boronic acid group of claim 12, wherein the biomolecule is an antibody.
